(19) United States
(12) Patent Application Publication
(10) Pub. No.: US 2022/0249152 A1
(43) Pub. Date: Aug. 11, 2022

(54) TREATMENT DEVICE FOR MEDICAL PROCEDURES

(71) Applicant: OLYMPUS MEDICAL SYSTEMS CORP., Tokyo (JP)

(72) Inventor: Yukihiko SHIMAMURA, Yoshikawa-shi (JP)

(73) Assignee: OLYMPUS MEDICAL SYSTEMS CORP., Tokyo (JP)

(21) Appl. No.: 17/579,730

(22) Filed: Jan. 20, 2022

Related U.S. Application Data
(60) Provisional application No. 63/146,895, filed on Feb. 8, 2021.
(60) Provisional application No. 63/146,901, filed on Feb. 8, 2021.

(51) Int. Cl. A61B 18/14

(52) U.S. Cl. CPC A61B 18/14; A61B 2018/0063

(57) ABSTRACT

Treatment device having vibration source, transmission rod, and detachable tip is equipped with ultrasonic transducers. Ultrasonic vibrations transmitted along the transmission rod to the detachable tip cause a treatment end of the tip, such as a blade, to vibrate at ultrasonic frequencies to cut and/or seal tissues. The detachable tip is connected at a location other than the vibration node or the antinode position. The detachable tip endures increased stresses associated with this location by decreasing the mass and sectional area relative to the increasing stress levels. The detachable tip connects to the transmission rod using a transmission surface and a biasing member, such as a leaf spring, that engages a detent located in the housing at the distal end of the transmission rod. Biased contact between the transmission surface and contact surface transmits both ultrasonic vibrations and high-frequency currents to the treatment edge of the detachable tip.

### RELATED APPLICATION DATA

This application is based on and claims priority under 35 U.S.C. § 119 to U.S. Provisional Application Nos. 63/146,895 and 63/146,901, each of which was filed on Feb. 8, 2021. The entire contents of each of these applications are incorporated herein by reference.

### FIELD OF DISCLOSURE

The present disclosure relates to a treatment device, particularly for medical procedures, having a detachable tip. The treatment device is equipped with an ultrasonic transducer including piezoelectric elements that convert electrical power into ultrasonic vibrations. The ultrasonic vibrations are then transmitted along the vibration transmission members of the device to the detachable tip. The detachable tip includes a treatment edge that vibrates at ultrasonic frequencies to conduct a medical procedure, such as cutting and/or sealing tissues, as well as serves as high frequency electrodes.

### BACKGROUND

In the discussion that follows, reference is made to certain structures and/or methods. However, the following references should not be construed as an admission that these structures and/or methods constitute prior art. Applicant expressly reserves the right to demonstrate that such structures and/or methods do not qualify as prior art against the present invention.

FIG. 26 is a figure of a treatment device disclosed in the related art (United States Patent Application Publication No. 2017/0224403A1). The related art surgical treatment apparatus 11 includes hand piece 12 and driving device 13 connected by cable 14. The driving device 13 supplies power into the ultrasonic transducer placed at gripping portion 15 and high-frequency current into the probe 27, which is included in treatment portion unit 31 and has a periphery that is covered by case 44. The driving device 13 also includes an operation display panel 18 for setting or displaying the output level corresponding to the push-buttons 26. A drawback of the related art treatment device is that the probe 27 and the hand piece 12 are connected together as a unit in such a way that detachment of the probe by the operator, e.g., in order to replace the probe 27 in case it is damaged or needs to be removed for reprocess purposes, is precluded. This configuration of the related art treatment device is problematic since the capability to replace the tip portion of the treatment device is desired for economic and sanitary purposes.

FIGS. 27A and 27B are figures of another treatment device disclosed in the related art (U.S. Pat. No. 10,258,362). The related art treatment device consists of blade 260, annular flange 244, junction 242, and a waveguide 228 including a proximal portion 230 and a distal portion 232. The junction 242 consists of a projecting portion 236 extending proximally from the first contact surface 234 and a recess 240 formed on a second contact surface 238. The projecting portion 236 and recess 240 are configured to assemble in a press fit or interference fit manner. As discussed in the same related art, as a matter of physics, the distal end of blade 260 is located at a position corresponding to an antinode associated with resonant ultrasonic vibrations communicated through waveguide 228 (i.e., at an acoustic antinode). In contrast, junction 242 and flange 244 are located at positions corresponding to a node associated with resonant ultrasonic vibrations communicated through waveguide 228 to reduce the stress load at the junction 242. A drawback of the related art treatment device is that the size of the detachable distal portion 232 is dependent on the length that is equivalent to the distance between the distal end of the portion 232 as an antinode and the proximal end of the portion 232 as a node associated with resonant ultrasonic vibrations, in this case from the distal end of blade 260 to junction 242. Because the location of the blade and the junction need to be placed based on the position of the nodes and/or antinodes of the ultrasonic vibrations, there are limitations in the structure the detachable distal portion and the treatment device, making it difficult to reduce the manufacturing costs.

### SUMMARY

Accordingly, there is a need for designing a treatment device with a tip capable of being detached from the treatment device at a location other than the antinode or node associated with resonant ultrasonic vibrations and/or for the size of the detachable tip to be minimized, which would substantially obviate one or more of the issues due to limitations and disadvantages of related art treatment device.

An object of the present disclosure is to provide an improved treatment device that provides an efficient design for the associated detachable tip compared to the related art. At least one or some of the objectives is achieved by the treatment device disclosed herein.

Additional features and advantages will be set forth in the description that follows, and in part will be apparent from the description, or may be learned by practice of the invention. The objectives and other advantages of the disclosed treatment device will be realized and attained by the structure particularly pointed out in the written description and claims thereof, as well as the appended drawings.

Embodiments of the disclosed treatment device comprise an ultrasonic transducer, a transmission rod with a proximal end and a distal end connecting to the ultrasonic transducer at the proximal end and configured to transmit energy, and a tip with a first end and a second end detachably attached to the distal end of the transmission rod at the first end. The tip includes a hollow portion and an incline portion, the incline portion configured to reduce the sectional area in a direction from the first end to the second end of the tip.

Embodiments of the disclosed treatment device also comprise an ultrasonic transducer, a transmission rod with a proximal end and a distal end the proximal end of the transmission rod connected to the ultrasonic transducer and configured to transmit energy and the distal end of the transmission rod including a housing and a rod transmission surface, and a tip having a tip transmission surface detachably mounted to the housing at the distal end of the transmission rod. The housing includes a detent and the tip includes a biasing member and when the tip is detachably mounted to the housing, the biasing member engages the detent to bias a tip transmission surface to be pushed against the rod transmission surface.

Embodiments of the disclosed treatment device further comprise a treatment device wherein the biasing member is a leaf spring.

Embodiments of the disclosed treatment device further comprise a treatment device wherein the tip and the transmission rod member are comprised of different materials.

Embodiments of the disclosed treatment device further comprise a treatment device wherein the tip is comprised of Ti-6AL-4V.

Embodiments of the disclosed treatment device further comprise a treatment device wherein the tip is comprised of SUS316L, or SUS630.

Embodiments of the disclosed treatment device further comprise a treatment device wherein the tip is comprised of a material having a Rockwell C hardness around 10 or higher.

Embodiments of the disclosed treatment device further comprise a treatment device wherein the transmission rod is comprised of Ti-6AI-4V.

Embodiments of the disclosed treatment device further comprise a treatment device wherein the transmission rod is comprised of SUS316L or SUS630.

Embodiments of the disclosed treatment device further comprise a treatment device wherein the treatment device may generate or transmit high frequency currents used in high frequency treatments.

Embodiments of the disclosed treatment device further comprise a treatment device wherein the treatment device may simultaneously perform treatment using ultrasonic frequencies and high frequency currents.

Embodiments of the disclosed treatment device further comprise a treatment device wherein the transmission rod is configured to transmit high-frequency energy.

Embodiments of the disclosed treatment device further comprise a treatment device wherein the tip includes a high frequency electrode.

Embodiments of the disclosed treatment device further comprise a treatment device wherein a cover is detachably mounted to cover the transmission rod.

Embodiments of the disclosed treatment device further comprise a treatment device wherein a cover is fixedly attached to the tip.

Embodiments of the disclosed treatment device further comprise a treatment device wherein the incline portion reduces the sectional area monotonically.

Embodiments of the disclosed treatment device further comprise a treatment device wherein the tip includes more than one hollow portion.

Embodiments of the disclosed treatment device further comprise a treatment device wherein the tip includes a bridge dividing the hollow portion.

Embodiments of the disclosed treatment device further comprise a treatment device wherein the length of the tip is less than 2/10 waveguide length.

Embodiments of the disclosed treatment device further comprise a detachable tip comprising a treatment edge, configured to perform ultrasonic treatments, located at the first end and a second end detachably connecting to the treatment device. The tip includes a hollow portion and an incline portion, the incline portion configured to reduce the sectional area in a direction from the first end to the second end of the tip.

Embodiments of the disclosed detachable tip further comprise a detachable tip wherein the tip is comprised of Ti-6AL-4V.

Embodiments of the disclosed detachable tip further comprise a detachable tip wherein the tip is comprised of SUS316L or SUS630.

Embodiments of the disclosed detachable tip further comprise a detachable tip wherein the tip is comprised of a material having a Rockwell C hardness around 10 or higher.

Embodiments of the disclosed detachable tip further comprise a detachable tip wherein the treatment edge is configured to perform treatments using high-frequency currents.

Embodiments of the disclosed detachable tip further comprise a detachable tip wherein the treatment edge includes a high frequency electrode.

Embodiments of the disclosed detachable tip further comprise a detachable tip wherein the treatment device may simultaneously perform treatment using ultrasonic frequencies and high frequency currents.

Embodiments of the disclosed detachable tip further comprise a detachable tip wherein a cover is fixedly attached near the proximal end.

Embodiments of the disclosed detachable tip further comprise a detachable tip wherein the incline portion reduces the sectional area monotonically.

Embodiments of the disclosed detachable tip further comprise a detachable tip wherein the tip includes more than one hollow portion.

Embodiments of the disclosed detachable tip further comprise a detachable tip wherein the tip includes a bridge dividing the hollow portion.

Embodiments of the disclosed detachable tip further comprise a detachable tip wherein the length of the tip is less than 2/10 waveguide length.

Embodiments of the disclosed treatment device further comprise a treatment device comprising an ultrasonic transducer, a transmission rod with a proximal end and a distal end, and a tip including a torque receiving surface at a first end and a treatment edge at a second end. The proximal end of the transmission rod is connected to the ultrasonic transducer and the tip is detachably connected to the distal end of the transmission rod to transmit energy generated by the ultrasonic transducer along a length of the transmission rod and to the treatment edge of the tip. Between the torque receiving surface at the first end and the treatment edge at the second end, the tip further includes an incline portion and one or more hollow portions. In a direction from the first end to the second end the incline portion reduces a sectional area of the tip and a junction between the tip and the distal end of the transmission rod is closer to an antinode of the treatment device than to a node of the treatment device.

Embodiments of the disclosed treatment device further comprise a treatment device wherein the tip is detachably connected to the distal end of the transmission rod by a threaded connection.

Embodiments of the disclosed treatment device further comprise a treatment device comprising an ultrasonic transducer means, a transmission means with a proximal end and a distal end connecting to the base means at the proximal end and configured to transmit energy, and a tip means with a first end and a second end detachably attached to the distal end of the transmission means at the first end. The tip means includes a hollow means and an incline means, the incline means configured to reduce the sectional area in a direction from the first end to the second end of the tip means.

Embodiments of the disclosed treatment device further comprise a treatment device wherein the contacting surfaces where the tip and the housing contacts during the seating procedure is coated with lubrication coating.

Embodiments of the disclosed treatment device further comprise a treatment device wherein the contacting surfaces where the tip and the housing contacts during the seating procedure is coated with durability coating.

Embodiments of the disclosed treatment device further comprise a treatment device wherein the coating material used in the tip and housing are different.

Embodiments of the disclosed treatment device further comprise a treatment device wherein the thickness of the coating in the tip and housing are different.

Embodiments of the disclosed treatment device further comprise a treatment device wherein the thickness of the coating in the tip is smaller than the housing.

Embodiments of the disclosed treatment device further comprise a treatment device wherein the contacting surfaces in which the tip and the housing is contacting each other after completion of the seating process is not coated.

Embodiments of the disclosed treatment device further comprise a treatment device wherein the contact of the tip transmission surface with the rod transmission surface is sufficient to transmit energy from the transmission rod to the tip.

Embodiments of the disclosed treatment device further comprise a treatment device wherein the longitudinal length of the tip covers or nearly covers an entire waveguide length associated with resonant ultrasonic vibration of the treatment device.

Embodiments of the disclosed treatment device further comprise a treatment device wherein the tip includes an abutting surface that mounts on the distal end surface of the transmission rod.

Embodiments of the disclosed treatment device further comprise a detachable tip comprising a treatment edge configured to perform ultrasonic treatments, a transmission surface mountable to a treatment device, a bridge connecting the treatment edge and the transmission surface, and a biasing member.

Embodiments of the detachable tip further comprise a detachable tip wherein the biasing member is a leaf spring.

Embodiments of the detachable tip further comprise a detachable tip wherein the biasing member includes a hole.

Embodiments of the detachable tip further comprise a detachable tip wherein the biasing member includes a notch.

Embodiments of the detachable tip further comprise a detachable tip wherein the biasing member includes a protruding portion.

Embodiments of the detachable tip further comprise a detachable tip wherein the tip is comprised of Ti-6AL-4V.

Embodiments of the detachable tip further comprise a detachable tip wherein the tip is comprised of SUS316L, or SUS630.

Embodiments of the detachable tip further comprise a detachable tip wherein the tip is comprised of a material having a Rockwell C hardness around 10 or higher.

Embodiments of the detachable tip further comprise a detachable tip wherein the tip includes a high frequency electrode.

Embodiments of the detachable tip further comprise a detachable tip wherein the tip is coated with lubrication coating.

Embodiments of the detachable tip further comprise a detachable tip wherein the tip is coated with durability coating.

Embodiments of the treatment device further comprise a treatment device comprising an ultrasonic transducer, a transmission means with a proximal end and a distal end, the proximal end of the transmission means connected to the base means and configured to transmit energy and the distal end of the transmission means including a housing means and a rod transmission means, and a tip means having a tip transmission means, detachably mounted to the housing means at the distal end of the transmission means. The housing means includes a detent means and the tip means includes a biasing means and when the tip means is detachably mounted to the housing means, the biasing means engages the detent means to bias a tip transmission means to be in contact with the rod transmission means.

Embodiments of the treatment device can be disassembled for replacement of parts and/or cleaning. In example embodiments, a reassembly method of a treatment device comprises cleaning a used treatment device, removing a tip from the used treatment device, the tip being connected to the used treatment device at a position away from an antinode position of vibration of a transmission rod, removing a cover that straddles the tip and the transmission rod, and attaching an unused tip and an unused cover.

Other systems, methods, features and advantages will be, or will become, apparent to one with skill in the art upon examination of the following figures and detailed description. It is intended that all such additional systems, methods, features and advantages be included within this description, be within the scope of the present disclosure, and be protected by the following claims. Nothing in this section should be taken as a limitation on those claims. Further aspects and advantages are discussed below in conjunction with the embodiments of the disclosed input device. It is to be understood that both the foregoing general description and the following detailed description of the disclosed input device are examples and explanatory and are intended to provide further explanation of the disclosed input device as claimed.

### BRIEF DESCRIPTION OF THE DRAWING

The following detailed description of preferred embodiments can be read in connection with the accompanying drawings in which like numerals designate like elements and in which:

FIG. 1 illustrates a treatment device that includes an ultrasonic transducer, a transmission rod, and a detachable tip according to an embodiment of the present disclosure.

FIG. 2 illustrates the treatment device and its relationship with stress level associated with resonant ultrasonic vibrations and displacement as a function of position.

FIG. 3 illustrates the transmission rod and detachable tip and its relationship with wavelength associated with resonant ultrasonic vibrations.

FIG. 4 is a magnified view of one embodiment of a detachable tip.

FIGS. 5A and 5B illustrates the changes in the sectional area of the detachable tip as a function of distance.

FIG. 6 illustrates an example of usage of the treatment device to treat a treating tissue.

FIG. 7 illustrates an embodiment of a cover associated with the treatment device.

FIG. 8 is a magnified view of another embodiment of a detachable tip

FIG. 9 graphically illustrates changes in sectional area of the detachable tip as a function of distance for the embodiment of a detachable tip shown in FIG. 8.

FIG. 10 illustrates an embodiment of detachable tip in use during a medical procedure.

FIG. 11 is magnified view of still another embodiment of a detachable tip.

FIGS. 12A-C are magnified views of additional embodiments of a detachable tip.

FIG. 13 illustrates a treatment device that includes an ultrasonic transducer, a transmission rod, and a detachable tip according to an embodiment of the present disclosure.

FIG. 14 illustrates the treatment device and its relationship with stress level and displacement associated with resonant ultrasonic vibrations as a function of position.

FIG. 15 illustrates the transmission rod and detachable tip and its relationship with wavelength associated with resonant ultrasonic vibrations.

FIGS. 16A, 16B, and 16C are magnified views of embodiments of a detachable tip.

FIGS. 17A, 17B, and 17C are magnified views of the distal end of the transmission rod illustrating an embodiment of the housing to connect with the detachable tip.

FIGS. 18A, 18B, and 18C illustrates the connection process of mounting the detachable tip into the housing of the transmission rod.

FIG. 19 illustrates the detachable tip seated in the housing of the transmission rod.

FIG. 20 illustrates the cover covering the transmission rod and the detachable tip.

FIGS. 21 and 22 illustrate another embodiment of the detachable tip in which the detachable tip has a spatula shape.

FIG. 23 illustrates an embodiment of detachable tip in use during a medical procedure.

FIGS. 24 and 25 illustrate another embodiment of the detachable tip in which the detachable tip has a blade like shape and a gripping function.

FIGS. 26 and 27A-B are figures from the discussed prior art.

Throughout all of the drawings, dimensions of respective constituent elements are appropriately adjusted for clarity. For ease of viewing, in some instances only some of the named features in the figures are labeled with reference numerals.

### DETAILED DESCRIPTION

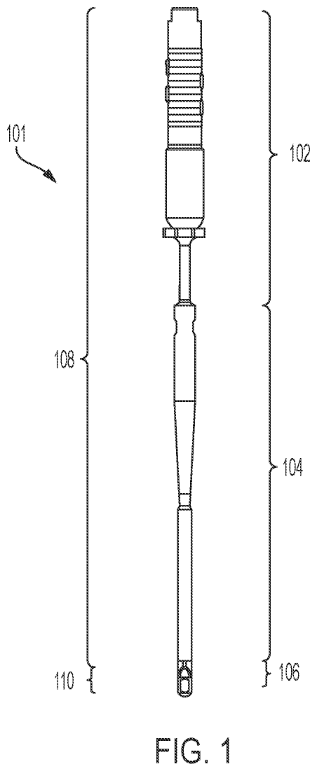

FIG. 1 is an illustration of a treatment device 101 including a bolt-clamped Langevin-type transducer (“BLT”) 102, a transmission rod 104, and a tip 106. The BLT 102 and transmission rod 104 constitute a reusable portion 108 and tip 106 constitutes a single use portion 110.

The BLT 102 is the portion of the treatment device 101 that the produces ultrasonic vibrations to be transmitted through the transmission rod 104 and tip 106 to the treatment area. BLT 102 is connected, through a cable or wirelessly, to a power source supplying electricity to the treatment device 101, including high-frequency currents used for high-frequency treatments. An ultrasonic transducer is included in the BLT 102 and is connected to the power source. The ultrasonic transducer includes piezoelectric elements that convert electrical power into ultrasonic vibrations. The ultrasonic vibrations are transmitted along the transmission rod 104 to the tip 106. The tip 106 includes a treatment edge that vibrates at ultrasonic frequencies to conduct medical procedures, such as incision and/or coagulation procedures. The tip 106 may also be used for medical procedures using high-frequency currents and serve as a high-frequency electrode. The tip 106 may also be used for medical procedures using both ultrasonic vibration and high-frequency currents and serve as a vibrating high-frequency electrode, also generally known as the combine mode.

The transmission rod 104 has a proximal end and a distal end and is connected to the BLT 102 at the proximal end. A function of the transmission rod 102 is to transmit ultrasonic vibrations and high-frequency currents to the tip 106, in order for the ultrasonic and high-frequency treatments to occur. The transmission rod 104 may be made from titanium alloys, such as Ti-6AI-4V or beta titanium alloys, stainless steels, such as SUS316L or SUS630, or other low dumping metal materials such as duralumin or amorphous metals to endure the stress incurred from use with ultrasonic frequencies and high-frequency currents.

The tip 106 has a first or proximal end and a second or distal end and is connected to the distal end of transmission rod 104 at its proximal end. The tip 106 is detachably connected using a screw, a threaded connection, or other jointing methods normally used to join metal objects together. The tip 106 may be made from titanium alloys, such as Ti-6AI-4V or beta titanium alloys, stainless steels such as SUS316L or SUS630, or other hard metal such as amorphous metals to endure the stress incurred from use with ultrasonic frequencies and high-frequency currents. Ideally, the hardness of the hard metal materials used for the tip 106 should be Rockwell C hardness around 10 or higher

The transmission rod 104 and tip 106 may be made from different materials. Using different materials may be beneficial in order to adjust the associated costs between the transmission rod 104, which is included in the reusable portion 108, and tip 106, which is included in the single use portion 110.

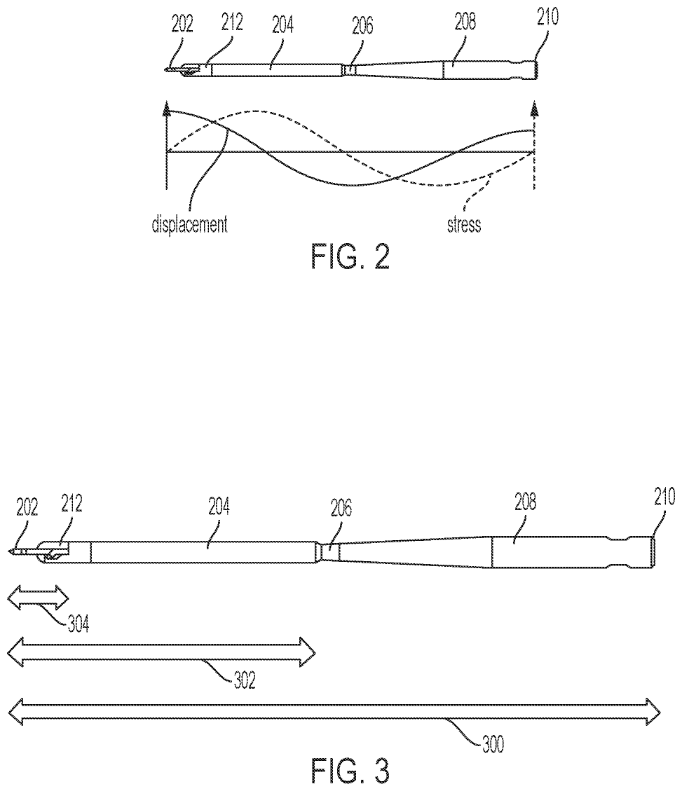

FIG. 2 illustrates the transmission rod 104 and a tip 106 and the displacement and stress levels (as a function of position along the length of transmission 104 and tip 106) in relation to resonant ultrasonic vibrations incurred by the treatment device 101. Under the general laws of physics, the resonant ultrasonic vibrations consist of a node position and an antinode position, the node position incurring the highest level of stress and antinode position incurring the lowest level of stress. As shown in FIG. 2, the displacement level decreases from the antinode 202 towards the node 204, reaches its lowest peak at antinode 206, and then increases towards node 208 and antinode 210. The stress level increases from antinode 202 and reaches its highest peak at node 204, and then decreases towards antinode 206 and reaches its lowest peak at node 208. As a matter of physics, the distal end of the tip 106 is placed at the antinode. The junction point 212 of the transmission rod 104 and the tip 106, which is susceptible to stress compared to other parts of the ultrasonic device, is placed in between the antinode 202 and node 204, where the stress level is relevantly high. Thus, there is a need to reduce the stress levels at the junction point 212.

FIG. 3 illustrates the relationship between the waveguide length of the resonant ultrasonic vibrations relative to transmission rod 104 and tip 106 of the treatment device 101. The waveguide length 300 is the entire wavelength of the resonant ultrasonic vibration starting from the antinode 202 to the adjacent antinode 204. The waveguide length 302 equals to half the length of the waveguide length 302, thus is half waveguide length. The length of the transmission rod 104 plus tip 106 in FIG. 3 equals to waveguide length 300, or 2 half waveguide length. Waveguide length 304 is the waveguide length equaling the distance between the distal end of tip 106 and the junction 212 of the transmission rod 104 and tip 106. The tip length 304 is ideally less than 2/10 of the waveguide length 300. This is because the stress level incurred by junction 212 increases as the wavelength 304 increases. The tip length 304 should be minimized in order to protect junction 212, which is more susceptible to damage from stress compared to the other portions of the treatment device 101, since it involves screws, thread connection, and other jointing mechanism used to join metal objects together.

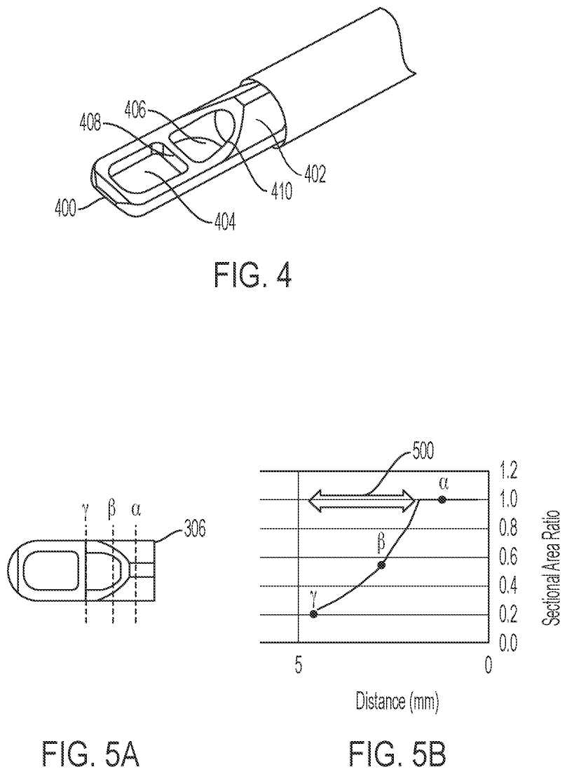

FIG. 4 shows a magnified view of tip 106 and junction 212 with transmission rod 104. The treatment edge 400 is located at the distal end of tip 106 and torque receiving surface 402, configured to provide a secure connection and the tightness of junction 212, is located near the proximal end of the tip 106. Two hollow portions 404 and 406 are located between the treatment edge 400 and torque receiving surface 402. The hollow portion 406 may serve to eliminate the incomplete thread portion, thereby shortening the length of tip 106, in case the junction 212 consists of a threaded connection. A bridge 408 separates the two hollow portions and, at the same time, provides structural strength to the tip 106. An incline portion 410 is placed between junction 212 and the treatment edge 400. The incline portion 410 forms a slope, which reduces the sectional area of the tip 106 in the direction from the proximal end to the distal end. This provides a transition between the torque receiving surface 402 and the treatment edge 400, with the proximal end of the incline portion 410 having a larger, in some embodiments, a 5× or larger, sectional area than the distal end of the incline portion 410.

FIG. 5A illustrates magnified top view of tip 106 shown in FIG. 4. The lines α, β, and γ represents the location on the tip 106 of the vertical cross-section associated with the measurement of the sectional area that is graphically presented in FIG. 5B. FIG. 5B graphically illustrates the reduction of the sectional area of incline portion 410. The horizontal axis represents the horizontal distance in millimeters (mm) from junction 212 to a specific point in tip 106. For example, the horizontal distance from the junction 212 to point α, signifying the location of line α, is roughly 1 mm. The vertical axis in FIG. 5B represents the sectional area in millimeter squared (mm²). FIG. 5B shows that the sectional area in the incline portion 410 reduces monotonically throughout the distance 500, roughly representing the length of the incline portion 410. The hollow portion 404,406 and incline portion 410 are designed to protect the tip 106 from the stress levels due to the resonant ultrasonic vibrations increasing towards the proximal end of the tip 106. The design strengthens the durability of the tip 106 by using the hollow portion 404,406, torque receiving surface 402 and incline portion 410 to reduce mass of tip 106, thereby reducing the associated stress levels.

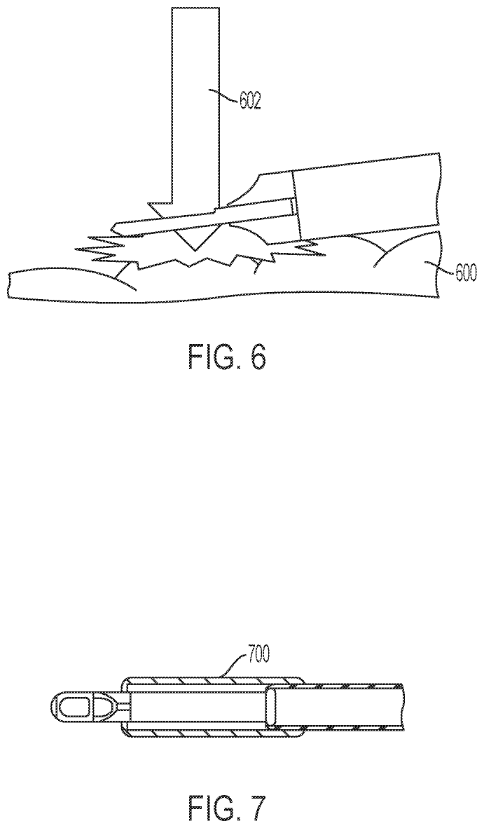

FIG. 6 is an illustration of the treatment device 101 in use with tissue 600. The tip 106, including the treatment edge 400, accesses the tissue 600 to conduct treatment (i.e. incision and coagulation to obtain hemostasis) using either ultrasonic vibration or high-frequency currents. During the coagulation procedure, physiological saline 602 is supplied to the tissue 600 to stabilize and cool the cauterization of the hemostatic site. The opening secured through the hollow portions 404,406 serves to stably supply the physiological saline 602 onto the hemostatic site.

FIG. 7 is an illustration of the treatment device 101 with a cover 700 encapsulating the distal end of the transmission rod 104 and a portion of tip 106. The cover 700 may be detachably mounted on the treatment device 101, or fixedly attached to the tip 106. The cover 700 is intended for single use and, therefore, when present is part of the single use portion 110 along with the tip 106. The purpose of the cover 700 is to protect the distal end of transmission rod 104 from damages incurred from the treatment procedure and for preventing dust intrusion purposes, as well as insulating the distal end portion of the transmission rod 104 during the high-frequency treatment procedures.

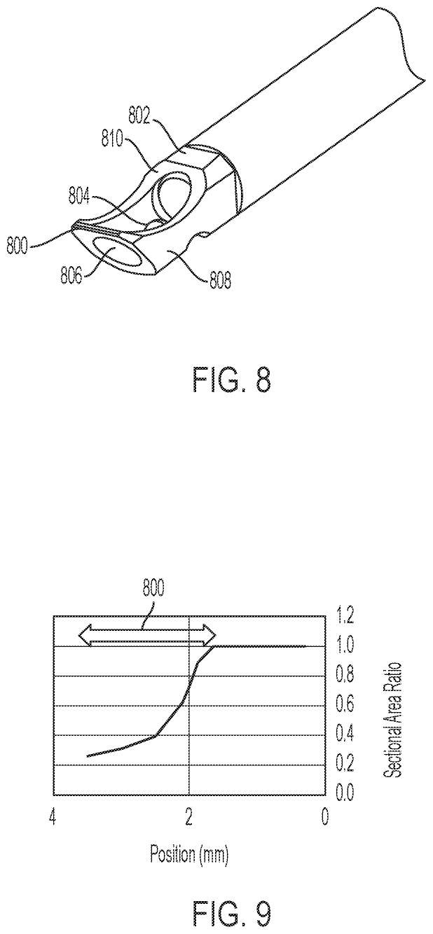

FIG. 8 illustrates another embodiment of tip 106. The treatment edge 800 is located at the distal end of tip 106 and torque receiving surface 802, configured to provide a secure the connection and the tightness of junction 212, is located near the proximal end of the tip 106. Two hollow portion 804 and 806 are placed between the treatment edge 800 and torque receiving surface 802. A bridge 808 separates the two hollow portions and, at the same time, provides structural strength to the tip 106. An incline portion 810 is placed between junction 212 and the treatment edge 800. The incline portion 810 forms a slope, which reduces the sectional area of the tip 106 in the direction from the proximal end to the distal end. This provides a transition between the torque receiving surface 802 and the treatment edge 800, with the proximal end of the incline portion 810 having a larger, in some embodiments, a 5× or larger, sectional area than the distal end of the incline portion 810.

FIG. 9 graphically illustrates the reduction of the sectional area of incline portion 810. The horizontal axis represents the horizontal distance in millimeters (mm) from junction 212 to a specific point in tip 106. The vertical axis in FIG. 9 represents the sectional area in millimeter squared (mm²). FIG. 9 shows that the incline portion 810 reduces the sectional area in a less monotone manner compared to the tip 106 shown in FIG. 4 and graphically illustrated in FIG. 5B. Whereas the change in sectional area in FIG. 5B occurs as a continuous curve, here the change in sectional area occurs in a series of linear steps, as the position changes through the distance 800, roughly representing the length of the incline portion 810.

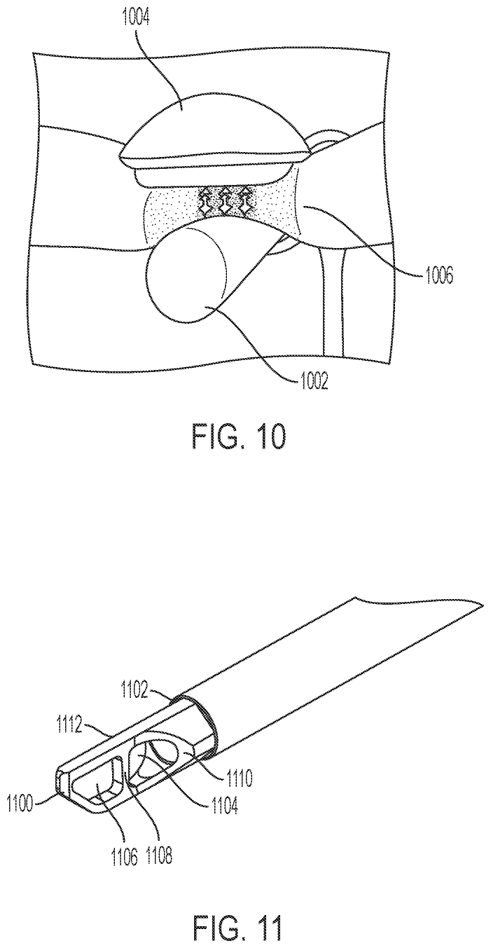

FIG. 10 illustrates an example usage of the ultrasonic probe 1002 similar to tip 106, combined with a grasping grip 1004 having a jaw type structure. The ultrasonic probe 1002 together with the grasping grip 1004 may grasp a treating tissue 1006 in between and perform treatment function such as incision and coagulation using either ultrasonic vibration or high-frequency currents.

FIG. 11 illustrates another embodiment of tip 106 designed to be used with the grasping grip 1004 described in FIG. 11. The treatment edge 1100 is located at the distal end of tip 106 and torque receiving surface 1102, configured to provide a secure connection and the tightness of junction 212, is located near the proximal end of the tip 106. Two hollow portions 1004 and 1006 are placed between the treatment edge 1100 and torque receiving surface 1102. A bridge 1108 separates the two hollow portions and, at the same time, provides structural strength to the tip 106. An incline portion 1110 is placed between junction 212 and the treatment edge 1100. The incline portion 1110 forms a slope, which reduces the sectional area of the tip 106 in the direction from the proximal end to the distal end. The upper edge 1112 may be used with a grasping grip 1004 described in FIG. 10 to grasp and perform treatment function such as incision and coagulation using either ultrasonic vibration or high-frequency currents.

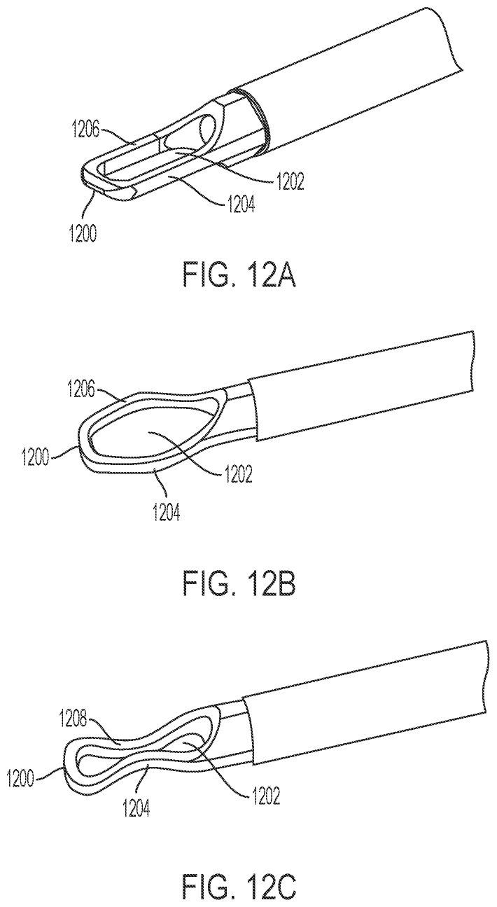

FIG. 12A illustrates another embodiment of tip 106. The treatment edge 1200 is located at the distal end of tip 106 and the tip 106 includes single hollow portions 1202. As further described in FIGS. 12A and 12B, by not placing a bridge, the side portions 1204 and 1206 vibrates by opening (FIG. 12B) and closing (FIG. 12C) at ultrasonic frequencies, which enables it to be used as treatment edge.

FIG. 12B illustrates the tip 106 where the treatment edge 1204 and 1206 is at an opened state. On the contrary, FIG. 12C illustrates the tip 106 where the treatment edge 1204 and 1206 is at a closed state. By repeating the open and close movement at an ultrasonic frequency, the treatment edge 1204 and 1206 may be used for incision and/or coagulation.

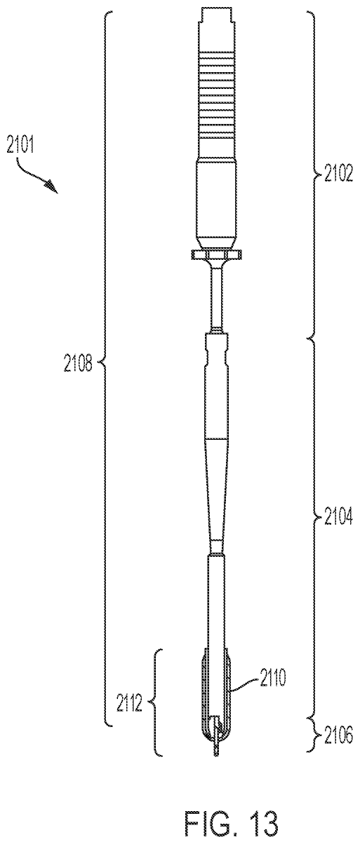

FIG. 13 is an illustration of a treatment device 2101 including a bolt-clamped Langevin-type transducer (“BLT”) 2102, a transmission rod 2104, and a tip 2106. The BLT 2102 and transmission rod 2104 constitute a reusable portion 2108 and tip 2106 constitutes a single use portion 2110.

The BLT 2102 is the portion of the treatment device 2101 that produces ultrasonic vibrations to be transmitted through the transmission rod 2104 and tip 2106 to the treatment area. BLT 2102 is connected, through a cable or wirelessly, to a power source supplying electricity to the treatment device 2101, including high-frequency currents used for high-frequency treatments. An ultrasonic transducer is included in the BLT 2102 and is connected to the power source. The ultrasonic transducer includes piezoelectric elements that convert electrical power into ultrasonic vibrations. The ultrasonic vibrations are transmitted along the transmission rod 2104 to the tip 2106. The tip 2106 includes a treatment edge that vibrates at ultrasonic frequencies to conduct medical procedures, such as incision and/or coagulation procedures. The tip 2106 may also be used for medical procedures using high-frequency currents and serve as a high-frequency electrode. The tip 2106 may also be used for medical procedures using both ultrasonic vibration and high-frequency currents simultaneously and serve as a vibrating high-frequency electrode, also generally known as the combine mode.

The transmission rod 2104 has a proximal end and a distal end and is connected to the BLT 2102 at the proximal end. A function of the transmission rod 2102 is to transmit ultrasonic vibrations and high-frequency currents to the tip 2106, in order for the ultrasonic and high-frequency treatments to occur. The transmission rod 2104 may be made from titanium alloys, such as Ti-6AI-4V or beta titanium alloys, stainless steels, such as SUS316L or SUS630, or other low dumping metal materials such as duralumin or amorphous metals to endure the stress incurred from use with ultrasonic frequencies and high-frequency currents.

The tip 2106 has a proximal end and a second or distal end and is connected to the distal end of the transmission rod 2104 at the proximal end. The tip 2106 is detachably connected using a mechanism incorporating a biasing member, such as a leaf spring, or elastic member including hole, notch, or a protruding portion as further described. The tip 2106 may be made from titanium alloys, such as Ti-6AI-4V or beta titanium alloys, stainless steels, such as SUS316L or SUS630 to endure the stress incurred from use with ultrasonic frequencies and high-frequency currents. Ideally, the hardness of the hard metal materials used for the tip 2106 should be Rockwell C hardness around 10 or higher.

The transmission rod 2104 and tip 2106 may be made from different materials. Using different materials may be beneficial in order to adjust the associated costs between the transmission rod 2104, which is included in the reusable portion 2108, and tip 2106, which is included in the single use portion 2110.

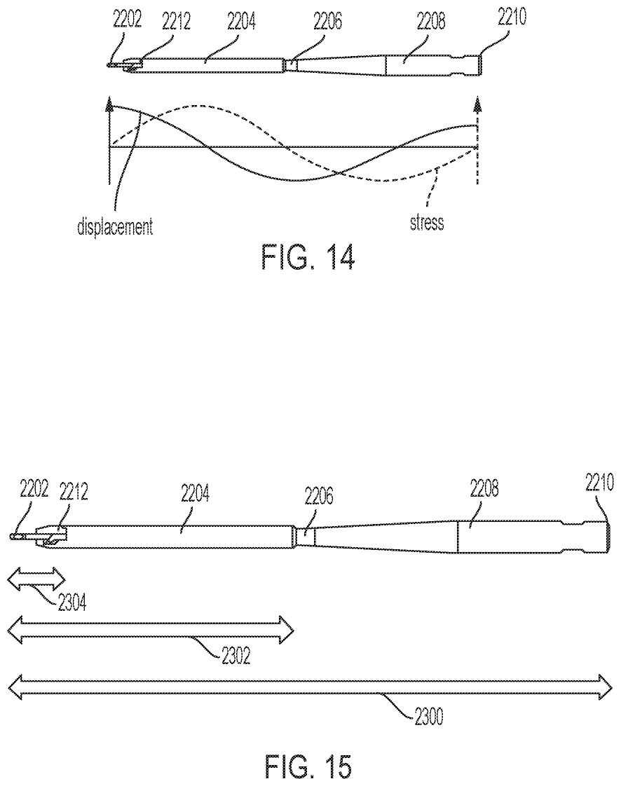

FIG. 14 illustrates the transmission rod 2104 and a tip 2106 and the displacement and stress levels (as a function of position along the length of transmission 2104 and tip 2106) in relation to resonant ultrasonic vibrations incurred by the treatment device 2101. Under the general laws of physics, the resonant ultrasonic vibrations consist of a node position and an antinode position, the node position incurring the highest level of stress and antinode position incurring the lowest level of stress. As shown in FIG. 14, the displacement level decreases from the antinode 2202 towards the node 2204, reaches its lowest peak at antinode 2206, and then increases towards node 2208 and antinode 2210. The stress level increases from antinode 2202 and reaches its highest peak at node 2204, and then decreases towards antinode 2206 and reaches its lowest peak at node 2208. As a matter of physics, the distal end of the tip 2106 is placed at the antinode. The junction point 2212 of the transmission rod 2104 and the tip 2106, which is susceptible to stress compared to other parts of the ultrasonic device, is placed in between the antinode 2202 and node 2204, where the stress level is relevantly high. Thus, there is a need to reduce the stress levels at the junction point 2212.

FIG. 15 illustrates the relationship between the waveguide length of the resonant ultrasonic vibrations relative to transmission rod 2104 and tip 2106 of the treatment device 2101. The waveguide length 2300 is the entire wavelength of the resonant ultrasonic vibration starting from the antinode 2202 to the adjacent antinode 2204. The waveguide length 2302 equals to half the length of the waveguide length 2302, thus is half waveguide length. The length of the transmission rod 2104 plus tip 2106 in FIG. 15 equals to waveguide length 2300, or 2 half waveguide length. Waveguide length 2304 is the waveguide length equaling the distance between the distal end of tip 2106 and the junction 2212 of the transmission rod 2104 and tip 2106. The tip length 2304 is ideally less than 2/10 of the waveguide length 2300. This is because the stress level incurred by junction 2212 increases as the wavelength 2304 increases. The tip length 2304 should be minimized in order to protect junction 2212, which is more susceptible to damage from stress compared to the other portions of the treatment device 2101, since it involves screws, thread connection, and other jointing mechanism used to join metal objects together.

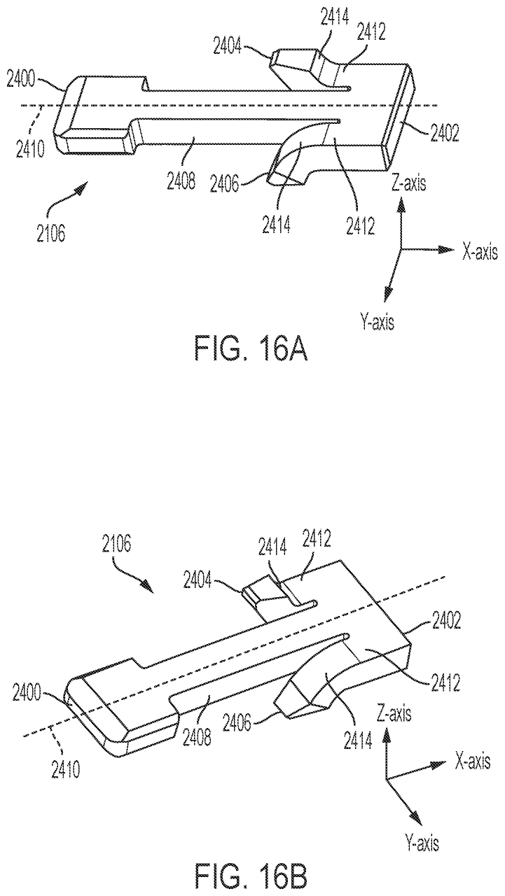

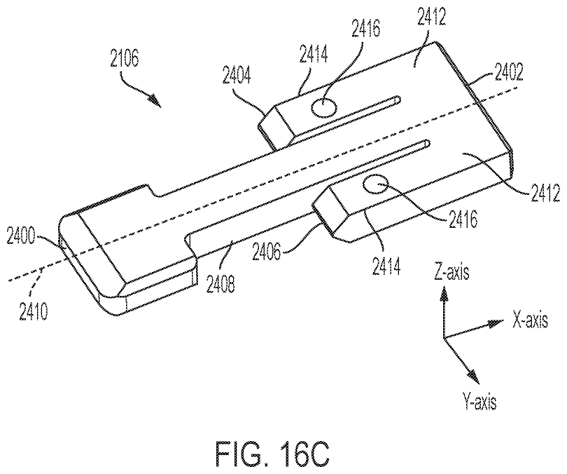

FIGS. 16A and 16B, each with separate perspective views, illustrate an embodiment of the tip 2106. The treatment edge 2400 is located at the distal end of tip 2106 and tip transmission surface 2402, configured to transmit vibration and high-frequency currents at junction 2212, is located at the proximal end of the tip 2106. A bridge 2408 connects the treatment edge 2400 to the tip transmission surface 2402 and is coincident to a longitudinal axis 2410 of the tip 2106. Biasing members, here shown as two leaf springs 2404 and 2406, are located between the treatment edge 2400 and tip transmission surface 2402 and are located on opposite sides of the longitudinal axis 2410. In addition, each of the biasing members 2404,2406 are to the respective side of the bridge 2408. A base section 2412 of each of the biasing members 2404,2406 is connected to body of the tip 2106 and the projecting portion 2414 of each of the biasing members 2404,2406 extends along a side of and is unattached to the bridge 2408. This projecting portion 2414 can be elastically compliant or elastically deformed, i.e., elastically moved in the Z-axis shown in FIGS. 16A and 16B. This elastic deformation pivots the projecting portions 2414 relative to their respective base sections 2412. In embodiments in which the tip is substantially planar (with the tip body including the bridge 2408 in a plane containing the longitudinal axis 2410, i.e., the plane containing the X-axis and Y-axis in FIGS. 16A and B), the elastic deformation moves the projecting portions 2414 into and out of the plane (i.e., in the Z-axis direction). In some embodiments, the projecting portions 2414 are formed with at least a part thereof out of the plane (i.e., the plane containing the X-axis and Y-axis in FIGS. 16A and B) when in the non-elastically deformed condition, in which case the elastic deformation moves at least this part into and out of the plane (i.e., in the Z-axis direction).

FIG. 16C illustrates another embodiment of the tip 2106. The treatment edge 2400 is located at the distal end of tip 2106 and tip transmission surface 2402, configured to transmit vibration and high-frequency currents at junction 2212, is located at the proximal end of the tip 2106. A bridge 2408 connects the treatment edge 2400 to the tip transmission surface 2402 and is coincident to a longitudinal axis 2410 of the tip 2106. Elastic members, here shown as 2404 and 2406, are located between the treatment edge 2400 and tip transmission surface 2402 and are located on opposite sides of the longitudinal axis 2410. In addition, each of the elastic members 2404,2406 are to the respective side of the bridge 2408. A base section 2412 of each of the elastic members 2404,2406 is connected to body of the tip 2106 and the projecting portion 2414 of each of the elastic members 2404,2406 extends along a side of and is unattached to the bridge 4208. This projecting portion 2414 can be elastically compliant or elastically deformed, i.e., elastically moved in the Z-axis shown in FIG. 16C. This elastic deformation pivots the projecting portions 2414 relative to their respective base sections 2412. Hole 2416 is formed on the projecting portions 2414 that is intended to receive a protruding member formed on the housing of the transmission rod 2104. The hole 2416 could be a notch or a protruding member to be fitted into a hole or a notch formed on the housing of the transmission rod 2104.

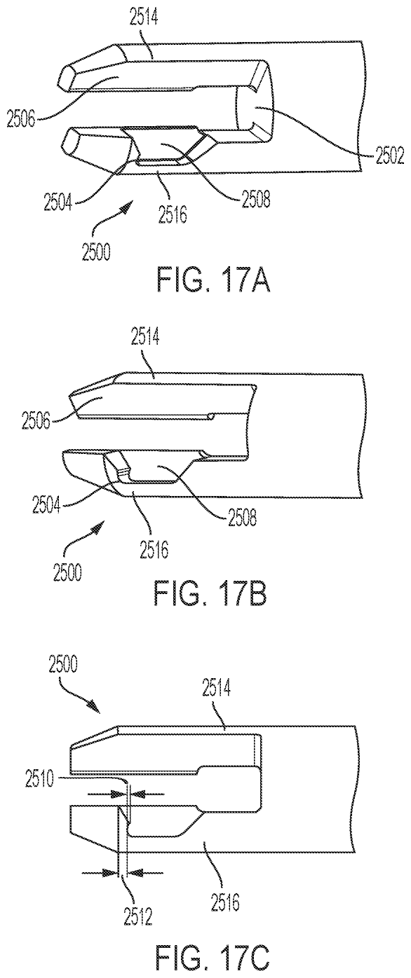

FIGS. 17A and 17B, each with different perspective views, show the distal end of the transmission rod 2104 with housing 2500, which is used to connect the transmission rod 2104 with tip 2106, which, when together, form junction 2212. When connected, the rod transmission surface 2502 is in contact with the tip transmission surface 2402. Such contact is sufficient to allow energy transmitted by the transmission rod 2104 to be conveyed to the tip 2106. In other words, the abutting contacting of the rod transmission surface 2502 and the tip transmission surface 2402 allows for energy transmitted by the transmission rod 2104 to propagate from the transmission rod 2104 to the tip 2106 sufficiently so that the treatment edge 2400 is operative. In addition, the biasing mechanism, such as the interaction of leaf springs 2404, 2406 with a respective detent 2504, form a compliant system that functions to fix the position of the tip 2106 within the housing 2500. Note that in some embodiments, both the biasing member on the tip 2106 and the legs 2514, 2516 of the housing 2500 can be elastically deformed, while in other embodiments one or the other of the biasing member on the tip 2106 and the legs 2514, 2516 of the housing 2500 can be elastically deformed. The walls 2504 and 2506 of the legs 2514, 2516 serve to maintain the rigidity of the housing 2500. FIG. 17C illustrates the spring detent stroke 2510 and spring bending stroke 2512 for engagement with leaf springs 2404 and 2406 in the compliant system that connects the tip 2106 to the housing 2500. The extent of spring detent stroke 2510 and spring bending stroke 2512, together with the material properties and size and shape of the leaf springs 2404,2406, relates to the strength of the bond between connection of transmission rod 2104 and tip 2106 at junction 2212.

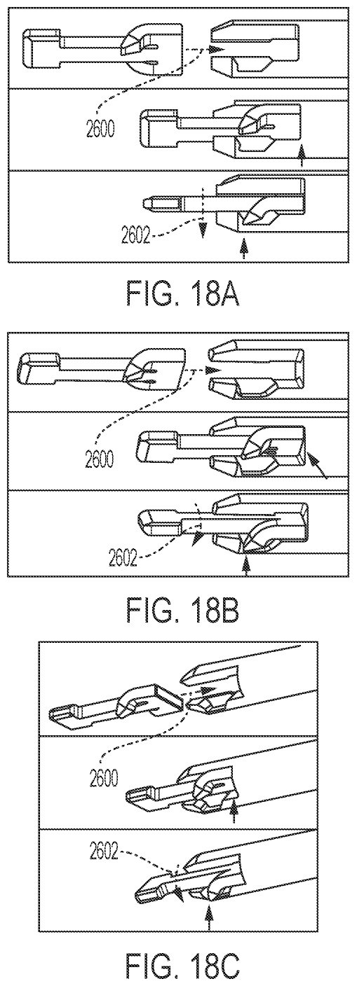

FIGS. 18A, 18B, and 18C illustrate the connection process of mounting the detachable tip 2106 into the housing 2500 to make the tip transmission surface 2402 contact the rod transmission surface 2502. The figures further show how, during the mounting process, the tip 2106 and housing 2500 are rotated (relative to each other and about the longitudinal axis 2410) in order to insert the tip 2106 into the space formed between the separate legs 2514,2516 and then how the tip 2106 is rotated relative to the housing so that the biasing members, such as leaf springs 2404,2406, are positioned in detent 2504. In this way, the tip 2106 and the transmission rod 2104 are connected to form junction 2212. In FIGS. 18A to 18C, arrows 2600 and 2602 show the direction of lateral and rotational movement of the tip 2106.

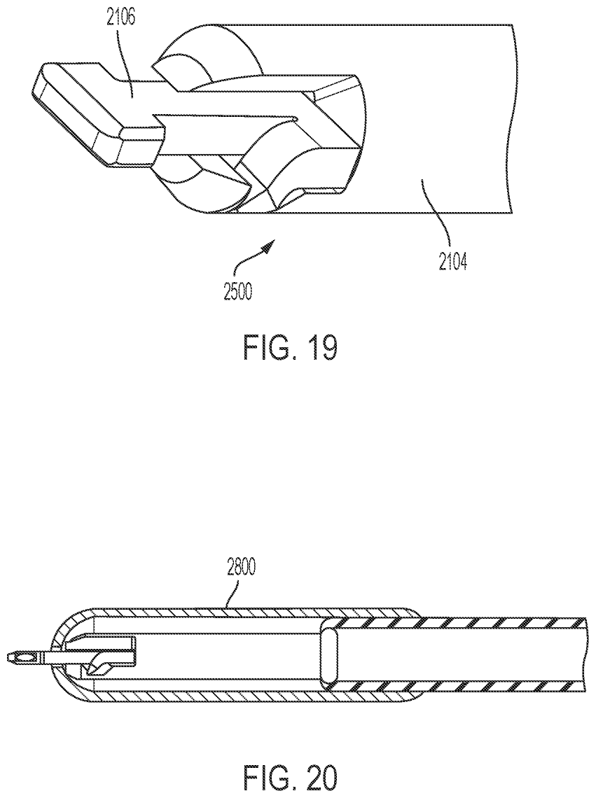

FIG. 19 illustrates the detachable tip 2106 seated in the housing of the transmission rod 2104 after the connection process illustrated in FIGS. 18A to 18C. Tip 2106 is seated within housing 2500 to form junction 2212 with tip transmission surface 2402 contacting the rod transmission surface 2502 and leaf spring 2404,2406 seated within detent 2504 to effectuate biasing force resulting from the compliant system, such as related to the strength of leaf spring 2404,2406.

Coating may be applied to the contacting surface of the tip 2106 and the housing 2500, such as the surface 2402, 2404, 2406, 2502, 2504, in order to improve sliding property and/or improve durability of the contacting portion of tip 2106 and housing 2500. A high durability/cost coating, such as titanium nitride or diamond-like-carbon, may be suitable for coating the contacting surface of transmission rod 2104 (i.e. 2502, 2504) since the transmission rod 2104 is part of the reusable portion 2108. A low durability/cost coating may be suitable for the contacting surface of tip 2106, (i.e. 2402, 2404, 2406), since the tip 2106 is part of the single use portion 2110. In order to achieve sufficient conductivity of high frequency current during the high frequency treatment procedure, the coating should have high permittivity or conductivity and should be coated at an appropriate thickness to achieve sufficient permittivity or conductivity. The thickness of the coating may be adjusted to wear out after the seating of the tip 2106 and housing 2500 in order to achieve sufficient permittivity or conductivity. In order to achieve sufficient transmittance of ultrasonic vibrations, the coating should have a hardness equal to or higher than the material of the tip 2106 and the housing 2500 and/or be thin enough to wear out after the seating of the tip 2106 and housing 2500. It may also be effective to coat the surfaces that comes in contact during the sliding process, but not coat the surfaces that comes in contact at the time the sliding process is completed and the tip 2106 seats within housing 500 in order to achieve sufficient conductivity of high frequency current and good transmittance for ultrasonic vibration.

FIG. 20 is an illustration of the treatment device 2101 with a cover 2800 encapsulating the distal end of the transmission rod 2104 and portion of tip 2106. The cover 2800 may be detachably mounted on the treatment device 2101, or fixedly attached to the tip 2106. The cover 2800 is intended for single use and, therefore, when present is part of the single use portion 2110 along with the tip 2106. The purpose of the cover 2800 is to protect the distal end of transmission rod 2104 from damages incurred from the treatment procedure and to prevent dust from intruding, as well as insulating the distal end portion of the transmission rod 2104 during the treatment procedures using high-frequency currents, including the procedure using the combine mode.

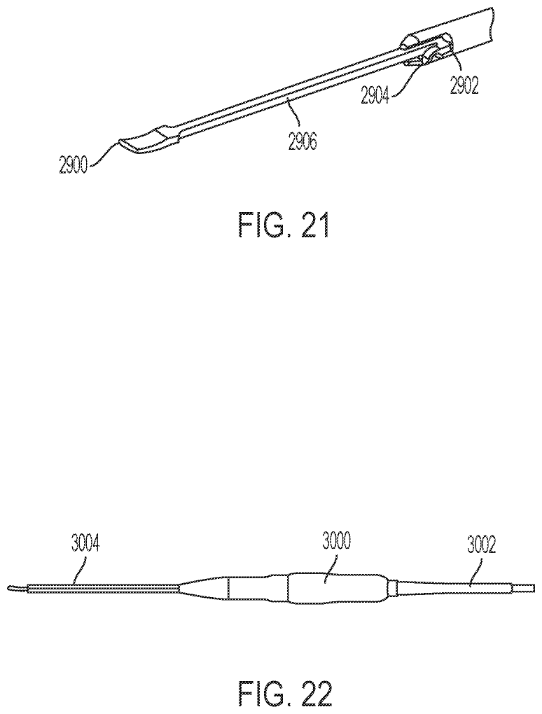

FIG. 21 illustrates another embodiment of tip 2106. The treatment edge 2900 is located at the distal end of tip 2106 and tip transmission surface 2902 and leaf spring 2904 are located near the proximal end of the tip 2106. A bridge 2906 is placed between the treatment edge 2900 and tip transmission surface 2902, connection the two portions while extending the tip 2106 in the longitudinal direction, which may allow the junction 2212 to be placed near the antinode where the stress level associated with the resonant ultrasonic vibration is low. The treatment edge 2900 is formed in the shape of a spatula for specific treatment purposes for ultrasonic treatments, high-frequency treatments, and treatments using the combine mode.

FIG. 22 illustrates an implementation of the spatula shaped treatment edge disclosed in FIG. 21. The base handle 3000 is connected to the transmission rod 2104 and tip 2106 to form the treatment device 2101, further connecting to power cable 3002. A cover 3004 is shown to cover the tip 2106 for protection purposes.

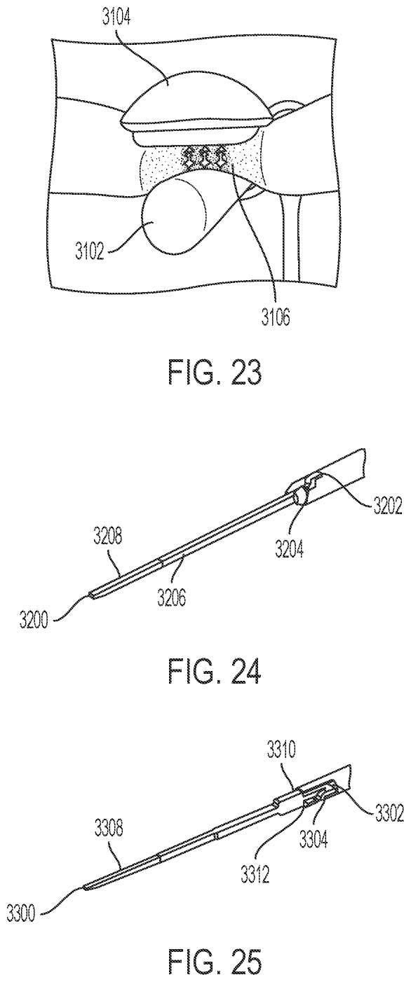

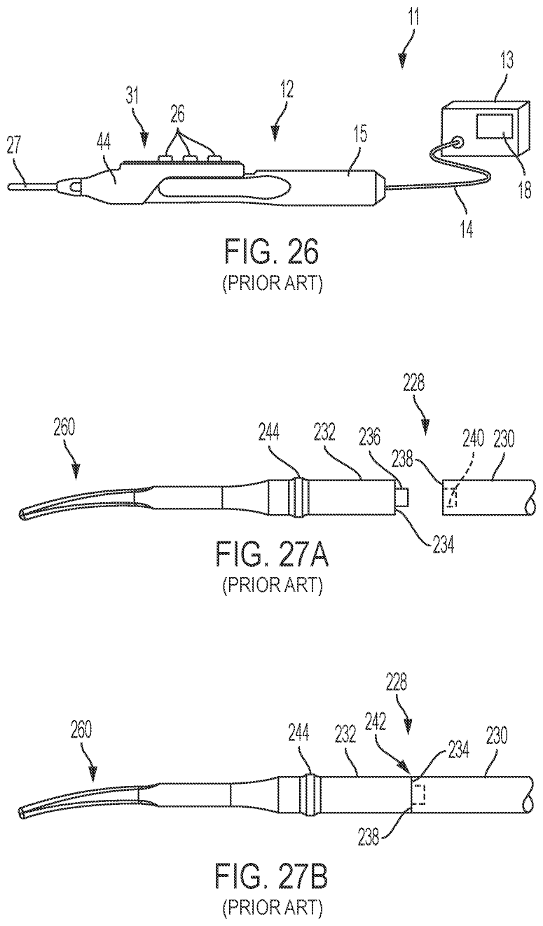

FIG. 23 illustrates an example usage of the ultrasonic probe 3102 similar to tip 2106, combined with a grasping grip 3104 having a jaw type structure. The ultrasonic probe 3102 together with the grasping grip 3104 may grasp a treating tissue 3106 in between and perform treatment function such as incision and coagulation using either ultrasonic vibration, high-frequency currents, or combine mode.

FIG. 24 illustrates another embodiment of tip 2106. The treatment edge 3200 is located at the distal end of tip 2106 and tip transmission surface 3202 and leaf spring 3204 are located near the proximal end of the tip 2106. A bridge 3206 is placed between the treatment edge 3200 and tip transmission surface 3202, connecting the two portions while extending the tip 2106 in the longitudinal direction, which may allow the junction 2212 to be placed near the antinode where the stress level associated with the resonant ultrasonic vibration is low. The upper edge 3108 is formed on the upper end of bridge 3206 and engages in the grasping function described in FIG. 23, performing the similar role as ultrasonic probe 3102.

FIG. 25 illustrates another embodiment of tip 2106. The treatment edge 3300 is located at the distal end of tip 2106 and leaf spring 3304 are located near the proximal end of the tip 2106. A bridge 3306 is placed between the treatment edge 3300 and proximal end surface 3302, extending the tip 2106 in the longitudinal direction. The upper edge 3308 is formed on the upper end of bridge 3306 and engages in the grasping function described in FIG. 23, performing the similar role as ultrasonic probe 3102. An abutting surface 3310 and 3312 is formed near the proximal end of the tip 2106, which abuts the distal end surface of the transmission rod 2104. The abutting surface 3310 and 3312 serves to transmit the ultrasonic vibration, as well as serving to increase the rigidity of the tip 2106 in case a downward force is applied during the grasping grip procedure. The location of the abutting surface 3310 and 3312 may be adjusted determining where antinode of the ultrasonic vibration would be placed within the tip 2106, in order to adjust the stress level associated with the resonant ultrasonic vibration applied to the junction 2212. For this embodiment, there should be gap between the proximal end surface 3302 and the distal end of the transmission rod 2104, because the abutting surfaces 3310 and 3312 act as transmission surfaces, and the proximal end surface 3302 should not act as transmission surface.

Although the length of the bridge and the geometry of the treatment edge in the embodiments illustrated and disclosed in connection to FIGS. 21, 24 and 25 differ from the embodiment illustrated and disclosed in connection with FIGS. 16A-C, 17A-C, 18A-C, and 19, other features are the same. For example, the biasing members, the compliant system, the housing including the legs and detent, and the process of mounting the tip in the housing are the same between the two embodiments.

Embodiments of the treatment device can be disassembled for replacement of parts and/or cleaning. In example embodiments, tip replacement can be made by a tip replacement operation that is performed according to (1) to (5) as follows. In (1), a used treatment device is cleaned, for example, by removing contaminants such as blood that may have adhered to the used treatment device. In (2), the used treatment device is disassembled. When disassembling, if a cover is provided as shown in FIGS. 7 and 20, the cover is removed. Thereafter, a used tip is removed from the transmission rod. In (3), assembly occurs including attaching an unused tip to the transmission rod. When the unused tip is a screw-type, e.g., attached to the transmission rod with a threaded connection, it is desirable to apply a predetermined torque to the unused tip by using a tool, such a torque wrench. In embodiments in which the unused tip is a biasing-type, e.g., attached to the transmission rod with a biasing member such as a leaf spring, the unused tip is assembled such that the biasing members contact a respective detent, for example, leaf springs 2404, 2406 are fixed to respective detent 2504. In (4), if a cover is used, the cover is attached to straddle the unused tip and the transmission rod. In (5), the assembled treatment device is cleaned, disinfected, and/or sterilized, as needed.

The above-disclosed tip replacement operation may be performed when replacing the tip of the treatment device that has been used, for example, in the hospital. The used treatment device may also be collected, for example by a vendor, to replace the tip of the collected treatment device.

A reassembly method of a treatment device comprises cleaning a used treatment device, removing a tip from the used treatment device, the tip being connected to the used treatment device at a position away from an antinode position of vibration of a transmission rod, removing a cover that straddles the tip and the transmission rod, and attaching an unused tip and an unused cover.

Although the present invention has been described in connection with preferred embodiments thereof, it will be appreciated by those skilled in the art that additions, deletions, modifications, and substitutions not specifically described may be made without department from the spirit and scope of the invention as defined in the appended claims.

What is claimed is:

1. A treatment device, comprising:
an ultrasonic transducer;
a transmission rod with a proximal end and a distal end connecting to the ultrasonic transducer at the proximal end and configured to transmit energy; and
a tip with a first end and a second end detachably attached to the distal end of the transmission rod at the first end,
wherein the tip includes a hollow portion and an incline portion, the incline portion configured to reduce the sectional area in a direction from the first end to the second end of the tip.

2. The treatment device according to claim 1, wherein the tip and the transmission rod member are comprised of different materials.

3. The treatment device according to claim 2, wherein the tip is comprised of Ti-6AL-4V, SUS316L, or SUS630.

4. The treatment device according to claim 2, wherein the tip is comprised of a material having a Rockwell C hardness around 10 or higher.

5. The treatment device according to claim 2, wherein the transmission rod is comprised of Ti-6AI-4V, SUS316L, or SUS630.

6. The treatment device according to claim 1, wherein the tip includes a high frequency electrode and the transmission rod is configured to transmit high-frequency currents.

7. The treatment device according to claim 1, wherein a cover is detachably mounted to cover the transmission rod and the cover is fixedly attached to the tip.

8. The treatment device according to claim 1, wherein the incline portion reduces the sectional area monotonically.

9. The treatment device according to claim 1, wherein the tip includes more than one hollow portion.

10. The treatment device according to claim 1, wherein the tip includes a bridge dividing the hollow portion.

11. The treatment device according to claim 1, wherein the length of the tip is less than 2/10 waveguide length.

12. The treatment device according to claim 1, wherein the first end of the tip is closer to an antinode position of vibration of the transmission rod than to a node position of the vibration of the transmission rod.

13. A reassembly method of a treatment device, comprising
cleaning a used treatment device;
removing a tip from the used treatment device, the tip being connected to the used treatment device at a position away from an antinode position of vibration of a transmission rod;
removing a cover that straddles the tip and the transmission rod; and
attaching an unused tip and an unused cover.

14. A treatment device, comprising:
an ultrasonic transducer;
a transmission rod with a proximal end and a distal end, the proximal end of the transmission rod connected to the ultrasonic transducer and configured to transmit energy and the distal end of the transmission rod including a housing and a rod transmission surface; and
a tip having a tip transmission surface, detachably mounted to the housing at the distal end of the transmission rod;
wherein the housing includes a detent and the tip includes a biasing member, and
wherein, when the tip is detachably mounted to the housing, the biasing member engages the detent to bias a tip transmission surface to be pushed against the rod transmission surface.

15. The treatment device according to claim 14, wherein the biasing member is a leaf spring.

16. The detachable tip according to claim 14, wherein the biasing member includes at least one of a hole, a notch, and a protruding portion.

17. The treatment device according to claim 14, wherein the contacting surfaces where the tip and the housing contacts during the seating procedure is coated.

18. The treatment device according to claim 17, wherein the coating material used in the tip and housing are different, the thickness of the coating in the tip and housing are different, or both the coating material used in the tip and housing are different and the thickness of the coating in the tip and housing are different.

19. The treatment device according to claim 17, wherein the contacting surfaces in which the tip and the housing is contacting each other after completion of the seating process is not coated.

20. The treatment device according to claim 14, wherein the tip includes an abutting surface that mounts on the distal end surface of the transmission rod.
